(19) United States
(12) Patent Application Publication
(10) Pub. No.: US 2020/0022907 A1
(43) Pub. Date: Jan. 23, 2020

(54) STABLE FORMULATIONS FOR CREAM

(71) Applicants: Max Spielberg, Beverly Hills, CA (US); David Johnson, Beverly Hills, CA (US)

(72) Inventors: Max Spielberg, Beverly Hills, CA (US); David Johnson, Beverly Hills, CA (US)

(21) Appl. No.: 16/040,985

(22) Filed: Jul. 20, 2018

(51) Int. Cl. A61K 9/00; A61K 31/045; A61K 47/10; A61K 47/46; A61K 47/36

(52) U.S. Cl. CPC A61K 9/0014; A61K 31/045; A61K 47/36; A61K 47/46; A61K 47/10

(57) ABSTRACT

The present invention is an organic roll-on pain formulation including Menthol—ethyl alcohol component premixed in a ratio of menthol: ethyl alcohol 1-6.0-8.0 along with a a carrier - preservative system consisting of components in a ratio of Glycerin: Aloe Vera Leaf Juice: Licorice Root Extract: Peppermint Oil: Xanthan Gum in a ratio of 1:0.3-0.4:0.006-0.007:0.3-0.4:0.10-0.15 each combined in water.

### SUMMARY OF THE INVENTION

The present invention relates to novel stabilization of liquid pharmaceutical and nutritional supplements.

In one embodiment, the present invention is a preservative system that contains only certified organic alcohol as the entire preservative system. By using a much larger percentage of alcohol in the product compared to conventional creams, the present invention has succeeded in formulating a product that does not need any other non-organic preservatives and does not need any other preservative except the organic alcohol. This formulation provided for the first certified organic cream, without using any synthetic preservatives.

In another embodiment, the invention is a formulation using a combination of a high percentage of certified organic alcohol, certified organic licorice root and certified organic glycerin as the preservative system. This formulation provided for the first certified organic cream, with an even longer shelf life, without using any synthetic preservatives.

In another embodiment, the invention addresses the need for a higher viscosity formulation that is compliant with the USDA organic regulations. This embodiment utilizes xanthan gum as a thickener and is allowed per USDA organic regulations as an allowed, natural, non-organic ingredient.

In another embodiment, the difficulty of utilizing menthol to correctly dissolve in the product, as menthol is a difficult ingredient to work with and often breaks into small parts. The invention discovered that a process utilized by adding the menthol to the alcohol, dissolve and subsequently solubilize the menthol in the alcohol, only to then add to the remainder of the formula. By adding the menthol into the alcohol separately from the rest of the formula, the process is able to prevent the menthol from breaking into small parts.

AS generally understood, the components of the present invention function as follows:

Alcohol is preservative and also solvent to hold ingredients.

Xanthan gum is thickener to give body so not runny.

Aloe is for cooling effect and hydration and healing and feel on skin.

Water is carrier to act as solvent and hold everything together.

Glycerin is humectant that holds water on skin and dries water.

Licorice root acts as preservative as well as skin conditioning.

Peppermint oil also gives a nice feel and for smell

In one embodiment, the present invention is a roll-on pain formulation comprising:
Menthol—ethyl alcohol component premixed in a ratio of
menthol: ethyl alcohol 1-6.0-8.0;
a carrier—preservative system consisting of components in
a ratio of Glycerin: Aloe Vera Leaf Juice: Licorice Root
Extract: Peppermint Oil: Xanthan Gum in a ratio of 1:0.3-0.4:0.006-0.007:0.3-0.4:0.10-0.15; and water q.s.

The formulation imparts a stable formulation exhibiting less than 100 Aerobic cfu/g and less than 100 Yeast and Mold cfu/g after ninety days storage at 40° C./75% relative humidity.

Also contemplated is a method for producing a formulation according to the present invention, said method consisting of the steps:

combining menthol and ethyl alcohol in a ratio of between 1:6.0-8.0;

adding the combined menthol and ethyl alcohol to water;

adding a carrier - preservative system consisting of components in a ratio of Glycerin: Aloe Vera Leaf Juice: Licorice Root Extract: Peppermint Oil: Xanthan Gum in a ratio of 1:0.3-0.4:0.006-0.007:0.3-0.4:0.10-0.15.

In one embodiment, the present invention is a roll-on pain formulation consisting of:
Menthol—ethyl alcohol component premixed in a ratio of menthol: ethyl alcohol 1-6.0-8.0;
a carrier—preservative system consisting of components in a ratio of Glycerin: Aloe Vera Leaf Juice: Licorice Root
Extract: Peppermint Oil: Xanthan Gum in a ratio of 1:0.3-0.4:0.006-0.007:0.3-0.4:0.10-0.15; and water q.s.

### DETAILED DESCRIPTION OF THE PREFERRED EMBODIMENT

It is widely recognized among formulations chemistry that a vast variety of variables interact. The chemical system still remains with unknown interactions that are discovered every day. the present invention has discovered two distinct formulations ratios that have been unknown until now to impart the desired stability.

It is known that formulation with menthol presents its own challenges. The present invention has discovered that an initial process step of dissolving menthol in a particularized ethyl alcohol ratio alleviates many of the formulation difficulties.

In one embodiment, a ratio of menthol: ethyl alcohol in 1:6.0-8.0 has demonstrated desirable results.

In another embodiment, a ratio of mental: ethyl alcohol: in 1:7.0-7.5 has also demonstrated a desirable result.

This initial menthol/ethyl alcohol mix is then combined with components having a particularized ratio.

Glycerin: Aloe Vera Leaf Juice: Licorice Root Extract:

Peppermint Oil: Xanthan Gum in a ratio of 1:0.3-0.4:0.006-0.007:0.3-0.4:0.10-0.15.

It should not be trivialized as providing percentage compositions or simple substitution from other products with similar percentage compositions.

Through extensive research and testing, the ratio disclosed herein exhibits synergistic effects unknown until this time.

As used herein, components disclosed refer to each of the following:

Ethyl Alcohol being Ethyl Alcohol, 190 Proof, CAS No. 64-17-5

Menthol being Hexahydrothymol; (lalpha,2beta,5alpha)-5- Methyl-2-(1-methylethyl)cyclohexanol, CAS No. 2216-51-5

Glycerin is as commonly understood to by 98.5-99.5% w/w grade.

Licorice Root Extract is the substantially pure extract generally known and characterized by a specific gravity of about 1.24-1.29 g/cm³ at 25° C. and a refractive index of about 1.4610 - 1.4790 at 25° C.

Peppermint oil is the oil extract as known, CAS No. 8006-90-4, with a specific gravity of about 0.90 g/cm³ at 25° C. and a refractive index of about 1.461 at 25° C.

Xanthan Gum used herein is (C₃₅H₄₉O₂₉)ₙ CAS 11138-66-2.

Aloe Vera Juice is Aloe Vera Fillet Juice Preservative Free containing 60-99.9 weight percent of Aloe Vera Juice, CAS 85507-69-3 and 0.1-1.0 weight percent of citric acid CAS 77-92-9 and q.s. water as needed.

In one embodiment, the present invention is formulated:

| | |
|---|---|
| Organic Menthol | 7.00-9.00% |
| Organic Aloe Barbadensis | 0.10-0.2% |
| (Aloe Vera) Leaf Juice | |
| Organic Ethyl Alcohol | 50.00-56% |
| Organic Glycerin | 3.00-3.20% |
| Organic Glycyrrhiza Glabra | 0.01-0.2% |
| (Licorice) Root Extract | |
| Organic Mentha Piperita | 0.85-1.15% |
| (Peppermint) Oil | |
| Xanthan Gum | 0.30-0.40% |
| Water q.s | |

In another embodiment, the formulation is as follows:

| | |
|---|---|
| Organic Menthol | 7.00% |
| Organic Aloe Barbadensis | 0.10% |
| (Aloe Vera) Leaf Juice | |
| Organic Ethyl Alcohol | 50.00% |
| Organic Glycerin | 3.08% |
| Organic Glycyrrhiza Glabra | 0.02% |
| (Licorice) Root Extract | |
| Organic Mentha Piperita | 1.00% |
| (Peppermint) Oil | |
| Xanthan Gum | 0.35% |
| Water q.s | |

TABLE 1
ACCELRATED STABILITY

| | | | Micro | | | | |
|---|---|---|---|---|---|---|---|
| | | | Total Aerobic | | | | |
| | | | Count: | | | | |
| | | Viscosity | NMT l00 cfu/g | Preservative | | | |
| | Appearance | RVT Spindle | Yeast & Mold | Challenge | | | Assay |
| | Translucent semi- | 3@50 rpm | NMT: 100 cfu/g | Meets USP | pH | Odor | Menthol 7.0% |
| Period/Condition | viscous Gel | TBD | Pathogens: Absent | Requirements | TBD | Menthol | (6.30%-7.70%) |
| Initial | Conforms | 640 cps | Passes | Passes | 7.82 | Conforms | 6.94% |
| Dec. 29, 2017 | | | | | | | |
| 1 Cycle F/T | N/A | N/A | N/A | N/A | N/A | N/A | N/A |
| 2 Cycle F/T | N/A | N/A | N/A | N/A | N/A | N/A | N/A |
| 3 Cycle F/T | Conforms | 540 cps | Passes | N/A | 7.70 | Conforms | 7.13% |
| 1 Month | Conforms | 720 cps | Passes | N/A | 7.49 | Conforms | 7.03% |
| 40° C./75% RH | | | | | | | |
| Jan. 29, 2018 | | | | | | | |
| 2 Month | Conforms | 820 cps | Passes | N/A | 7.02 | Conforms | 6.90% |
| 40° C./75% RH | | | | | | | |
| Feb. 29, 2018 | | | | | | | |
| 3 Month | Conforms* | 648 cps | Passes | Due Date: | 6.75 | Conforms | 7.06% |
| 40° C./75% RH | | | | May. 6, 2018 | | | |
| Mar. 29, 2018 | | | | | | | |

TABLE 2
ROOM TEMPERATURE STABILITY

| | | | Micro | | | | |
|---|---|---|---|---|---|---|---|
| | | | Total Aerobic | | | | |
| | | | Count: | | | | |
| | | Viscosity | NMT 100 cfu/g | Preservative | | | |
| | Appearance | RVT Spindle | Yeast & Mold | Challenge | | | Assay |
| | Translucent semi - | 3@50 rpm | NMT: 100 cfu/g | Meets USP | pH | Odor | Menthol 7.0% |
| Period/Condition | viscous Gel | TBD | Pathogens: Absent | Requirements | TBD | Menthol | (6.30%-7.70%) |
| Initial | Conforms | 640 cps | Passes | Passes | 7.82 | Conforms | 6.94% |
| Dec. 29, 2017 | | | | | | | |
| 3 Month | Conforms* | 612 cps | Passes | N7A | 7.36 | Conforms | 7.06% |
| 25° C./60% RH | | | | | | | |
| Mar. 29, 2018 | | | | | | | |

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

What is claimed:

1. A roll-on pain formulation comprising:
Menthol - ethyl alcohol component premixed in a ratio of menthol: ethyl alcohol 1-6.0-8.0;
a carrier—preservative system consisting of components in a ratio of Glycerin: Aloe Vera Leaf Juice: Licorice Root Extract: Peppermint Oil: Xanthan Gum in a ratio of 1:0.3-0.4:0.006-0.007:0.3-0.4:0.10-0.15; and water.

2. The formulation of claim 1, wherein said formulation imparts a stable formulation exhibiting less than 100 Aerobic cfu/g and less than 100 Yeast and Mold cfu/g after ninety days storage at 40° C./75% relative humidity.

3. A method for producing a formulation according to claim 1, said method consisting of the steps:
combining menthol and ethyl alcohol in a ratio of between 1:6.0-8.0;
adding the combined menthol and ethyl alcohol to water;
adding a carrier—preservative system consisting of components in a ratio of Glycerin: Aloe Vera Leaf Juice: Licorice Root Extract: Peppermint Oil: Xanthan Gum in a ratio of 1:0.3-0.4:0.006-0.007:0.3-0.4:0.10-0.15.
